(19) United States
(12) Patent Application Publication
(10) Pub. No.: US 2010/0240887 A1
(43) Pub. Date: Sep. 23, 2010

(54) New methods of producing HHT

(76) Inventor: Yaguang Liu, (US)

Correspondence Address: YAGUANG LIU, 22 Sunrise Bay Blvd., Little Egg Harbor, NJ, 08087, US

(21) Appl. No.: 12/383,273

(22) Filed: Mar. 23, 2009

(51) Int. Cl. C07D 493/06; A61K 31/55

(52) U.S. Cl. 540/581; 514/214.03

(57) ABSTRACT

A safe pharmaceutical composition for treatment of cancer contains Homoharringtonine (HHT). The new methods of process for producing HHT include culture plant tissue and semisynthesis.

### FIELD OF THE INVENTION

A safe pharmaceutical composition for treatment of cancer contains Homoharringtonine (HHT). The new methods of process for producing HHT include culture plant tissue and semisynthesis.

### DESCRIPTION BEFORE ART

The remarkable clinical efficacy of Homoharringtonine (HHT) resulting in lot of observations of complete remission of leukemia and other solid cancer in human being since 1988. Recently, research articles reported that the HHT efficacy in glaucoma, inhibition of Hepatities B virus replication and using in bone marrow transplantation. For example, the University of Texas M.D. Anderson Cancer Center and National Cancer Institute reported that “Ninety-two percent of patients achieved CHR with HHT.” [Susan O'Brien, at al.; Sequential homoharringtonine and interferon-α in the treatment of early chronic phase chronic myelogenous leukemia; Blood, Vol 93, No 12 (June 15), 1999: pp 4149-4153]. Another article reported that “the median number of days on HHT per month was 2 days with a median follow-up of 26 months; the estimated 2-year survival rate was 90%.” (Susan O'Brien, at al.; Simultaneous homoharringtonine and interferon-α in the treatment of patients with chronic-phase chronic myelogenous leukemia; American Cancer Society; Apr. 1, 2002, Vol 94, No. 7).

On Nov. 8, 1988, U.S. Pat. No. 4,783,454 titled Process for producing harringtonine and homoharringtonine disclosed the technique of isolation of a purified HHT from bark of Cephalotaxus. However, the natural source of Cephalotaxus is very limited. Trees of Cephalotaxus grow slowly. Bark of Cephalotaxus has very low content of HHT. Extracting HHT from bark of Cephalotaxus the yield was about 0.02% only. More important to harvest bark of Cephalotaxus will kill and destroy trees. Supply of HHT is very short now. Therefore, it is necessary to find a new manufacturing method.

### DETAILED DESCRIPTION

Great progress has been made in research on Homoharringtonine (HHT) production and on future generation HHT drug since 1988. For example, the University of Texas M.D. Anderson Cancer Center and National Cancer Institute reported that “Ninety-two percent of patients achieved CHR with HHT.” Another article reported that “the median number of days on HHT per month was 2 days with a median follow-up of 26 months; the estimated 2-year survival rate was 90%.”

The good clinical results of HHT in treating cancer brought to the major problem, which is the supply of HHT both short term and long term. It is apparent that a huge amount of bark of Cephalotaxus is needed for collection, extraction and purification of HHT. It is clear that due to the slow growth of the trees of Cephalotaxus, which is a nature source of HHT, and the killing of trees by harvesting bark is not a sustainable resource for HHT production.

Present invention disclosed new methods for producing HHT. The new methods of producing HHT are shown as follows.

1. Tissue Culture (Plant Cell Culture):

Culture manipulation to promote secretion of HHT is a new way for an extracellular product HHT. The biosynthetic methods can yield more HHT through precursor of HHT feeding. The production of HHT increased significantly after the addition of the precursors and special biochemical agents. Content of precursor of HHT abounds in tree and it is very cheap. The present methods include several significant developments in technique of culture plant tissues that are
(a) yields of HHT selected from rapid growth, resistance to infections organisms; and
(b) HHT can excrete into media.

Traditional method of plant culture is very difficult to overcome the problem of high cost. Therefore, traditional method appears too long to have commercial value. HHT is secondary metabolite of Cephalotaxus. Secondary compound acts in defense against the harmful effects of toxins, carcinogens or mutagens found in the plant. In fact, traditional method is very difficult to increase HHT contenting in plant tissues. The present new method uses a special biochemical agent for increasing content of HHT and more easily to purify HHT from other metabolites.

More important is that the key of the present new technique for producing high content of HHT in plant cell culture is to increase production of HHT by directed fermentation through precursor of HHT feeding. The present new methods are used special metabolite of Cephalotaxus for markedly enhance production of HHT. Therefore, the present invention disclosed a new source for the long term of producing HHT.

2. Using Precursor of HHT:

Recent research's results have established that direct production of HHT from its precursor and advances in biosynthetic understanding for HHT metabolism. Biosynthesis or semisynthesis of HHT from major nonactivity ingredients is well established through great advances in special biochemistry reactions. Using precursor of HHT for semisynthesis and increase of production in plant cell culture are new developing methods for producing HHT.

3. Using Leaves:

Our new method use leaves of tree of Cephalotaxus not use the bark. So far, the extraction of HHT is used bark. The leaves are harvested from the trees of Cephalotaxus, which grow in mountains of South China. The natural source of leaves is very abundance. The new methods do not use bark. Therefore, it can avoid destroy trees. The natural source of Cephalotaxus tree is very limited and slow growing. In fact, bark of Cephalotaxus has very low yield of HHT. The yield of HHT from Cephalotaxus bark is about 50-100 mg/kg of dried bark. The present new method, therefore, has a great economic and environmental value.

4. Semisynthesis:

HHT has received important chemical studies particularly in regard to structure and anticancer activity relationship and semisynthesis.

A great progress in biochemistry allows semisynthesis to use precursor of HHT from leaves of Cephalotaxus and to produce HHT. The total chemical synthesis of HHT appears too long to have commercial value too. Semisynthesis method can yield a high efficient conversion of precursor to HHT. It is other better biological source for manufacturing HHT. This new method uses closing chemical analogues to convert to HHT. This analogue is produced from leaves or other organ of Cephalotaxus. The present invention disclosed that new methods and techniques of manufacturing HHT could avoid chopping down Cephalotaxus trees which governmental environmentalists are trying to have declared a threatened species.

5. Using Taxol Residual

The anticancer drug Taxol is the most promising new chemotherapeutic agents that developed for cancer treatment in the past twenty years. Taxol has a unique mechanism of action. It has been shown to promote tubulin polymerization and stabilize microtubules against depolymerization. The FDA approved the clinical use of Taxol for several types of cancer. So far, annual sales of Taxol are more than $2 billion in market. Taxol is extracted from bark or leaves of an evergreen tree named Taxus species including Taxus brevifolia (or called Pacific yew). After Taxol has been extracted from bark or leaves, all residual materials of Taxus brecifolia named Taxus residual, which are waste.

Both taxol and HHT can be extracted from yew tree. The content of taxol is less than 0.01% in yew tree. The content of HHT in yew tree is about 0.01% -0.22%. The content of HHT is much higher than content of Taxol. Taxol extracted from bark of yew is difficult and expensive. One reason is that the presences of closely related congeners are similar to Taxol. A major congener is Cephalomannine (CPM), which is a waster of process in manufacturing of Taxol.

The chemical and physical characters are very close between Taxol and Cephalomannine (CPM).

CPM characterized by the same ring structure as Taxol and distinguishes from them only in C-13 ester structure. The present invention disclosed that CPM and related derivative are used to produce HHT.

The following specific examples will provide detailed illustrations of methods of producing relative drugs, according to the present invention and pharmaceutical dosage units containing demonstrates its effectiveness in treatment of cancer cells. These examples are not intended, however, to limit or restrict the scope of the invention in any way, and should not be construed as providing conditions, parameters, reagents, or

### EXAMPLE 1

### Production of HHT by Culture Cells

So far, HHT is extracted from bark and skins of Cephalotaxus species. However, growth of Cephalotaxus species is very slow and concentration of HHT in plant is extremely low. Furthermore, it is difficult to harvest the plants because of their low propagation rate and the danger of drastic reduced in plant availability. Also, cost of total chemical synthesis of HHT is very expensive and is not available for commerce now. For the reasons given above it is more difficult to obtain Cephalotaxus on a large scale for long time. Therefore, Cephalotaxus cell cultures are one of best methods for obtaining HHT. In this present invention, special elicitation is disclosed and it will significantly increase production of HHT.

The methods of cell and tissue culture are disclosed as below.

Parts of bark, stems, leaves, or roots of Cephalotaxus species were surface disinfected by treatment in 70% ethanol for 10 minutes and followed by 0.1 HgCl₂ for 3 minutes. Plant materials were washed five times for 10 minutes each by sterilized water. Parts of plant were cut into small pieces (0.5-1 mm) and put pieces to Murashige and Skoog's (MS) medium and supplemented with derivative of new active ingredient of phylum mycota (IPM), precursor of HHT which is a derivative of Cephalotaxus (CEP), tyrosine (TYR) naphthaleneacetic acid (NAA), Kinetin (3 mg/L), and 3% sucrose (w/v). PH of medium was adjusted to 5.7˜5.8. Agar (10 g/L) added to medium. Callus tissues are collected from agar media and suspension cultured cells were harvested by filtration and cultured in MS medium.

The cultures were kept in a culture room at 26° C.±1° C. Friable callus tissues were obtained. The callu was inoculated into 4 L of MS liquid medium containing sucrose, derivative of CEP, PHE, TYR, NAA and Kinetin. Then callus tissues were cultivated 26° C. for 35 days on rotary shaker operated at 120 rpm in the dark. Cells were subcultured into fresh medium of same composition every 2 weeks and maintained at 120 rpm at 26°±1° C. Packed cell volume (PCV), fresh weight (FW), dry weight (DW), concentration of HHT and concentration of sugar were determined every 5th day. The cells were harvested and dried.

In general, callus and suspension cultures of cephalotaxus species grow very slow and no production of free or esterified HHT. However, according to the present invention, addition of IPM to cultures cause a drastic increasing in HHT after 30 days of incubation. For example, in control group (no IPM), HHT in cultured cells is 0.020 mg/g dry weight, but in treatment group (addition of IPM) HHT is about 0.050 mg/g dry weight. Therefore, IPM can increase 250% of content of HHT. It has resulted in plant cell culture systems that producing HHT at concentration higher than those produced by the mother plant. The production of HHT increases significantly after the addition of precursors (CEP). Addition of CEP can increase HHT. Obviously, the present invention provided a new commercial and economic method for producing HHT. The IPM and precursors (CEP) play key role in cultured cells.

### EXAMPLE 2

### Semi-Synthesis of HHT

HHT shows a significant inhibitory activity against leukemia and other cancer. Concentration of HHT, however, has only 0.01% in natural sources. Cephalotazine (CEP) is major alkaloids present in plant extracts and the concentration of Cephalotaxus has about 1%. Therefore, concentration of CEP is about 100 times higher then HHT in nature plant sources. But CEP is inactive. For the reason given above, semisynthesis of HHT from CEP will increase huge natural sources of HHT.
(1) Extraction of CEP

10 kg of dried stems or leaves or roots of Cephalotaxus species were milled, placed in a percolator, along 80 L of 95% of ethanol, and allowed to stand 24 hours. The ethanol was recovered under reduced pressure (below 40° C.). 20 L of 5% tartaric acid was added to concentrated ethanol solution. The ammonia water was added to the acidic solution and adjusted pH to 9. The solution of pH 9 was filtered and yielded a filtrate. The filtrate was extracted with CHCl₃. CHCl₃ was recovered under reduced pressure and residue was obtained. The residue was chromatographed packed with alumna and eluted by CHCl₃-MeOH (9:1). Eluate was concentrated under reduced pressure. Residue was dried under vacuum. The product is CEP.
(2) Semisynthesized HHT from CEP

Materials and Methods

Melting points were determined on a Fisher-Johns apparatus. Infrared spectra were obtained on a Perkin-Elmer 567 infrared spectrophotometer or on a Beckman 4230 IR spectrophotometer. Peak positions were given in cm⁻¹. The IR spectra of solid samples were measured as potassium bromide dispersions, and the spectra of liquids were determined in chloroform or carbon tetrachloride solutions. NMR spectra were measured on a Varian A-60, Perkin-Elmer R-32, Varian EM-390, or Brüker WH-90 NMR spectrometer. Chemical-shift values were given in parts per million downfield from Me₄Si as an internal standard. Mass spectra were run on an AE1 MS-12 Finnigan 3300, or CEC21-110B mass spectrometer.

Preparative thin-layer chromatography was accomplished using 750-μm layers of aluminum oxide HF-254 (type E), aluminum oxide 60 PF-254 (type E), silica gel HF-254 (type 60 PF-254), or silica gel GF-254. Visualization was by short-wave ultraviolet light. Grace silica gel, Grade 923, and Woelm neutral aluminum oxide, activity III, were used for column chromatography. Analytical thin-layer chromatography was run on plastic sheets precoated with aluminum oxide F-254 neutral (type T), 200-μm thick, and on Polygram Sil G/UV254 (silica gel), 250 μm on plastic sheets. Visualization was usually by short-wave ultraviolet light, phosphomolybdic acid, or iodoplatinate.

Preparation of α-Ketoester-Harringtonine

1 g of Benzene-α-acetone Na was put into 10 L of benzene. Mixture was stirred at room temperature then was dissolved in 10 L of pyridine and stirred at 0° C. Oxalic chloride was added from a dropping funnel to solution of pyridine. Stirring was continued while the solution warmed to room temperature and stand overnight. Excess reagent was removed. This solution was dissolved in CH₂Cl₂ and cooled to near 0° C. in an ice water bath. 5 g of CEP, 2.5 L of CH₂Cl₂ and 2.5 L of pyridine were added to cold CH₂Cl₂ solution. Manipulations were done in a dry N₂ atmosphere and all glassware heat-dried just before use. The suspension was stirred at room temperature and overnight. The mixture was washed with 10% Na₂CO₃ and saturated aqueous NaCl, then dried with auhydrous magenesium sulfate, and filtered and the solvents were removed in vacuo. Evaporation provided as an amorphous solid α-ketoester-harringtonine (mp 143˜145° C.).

Semi-Synthesis of HHT

10 L of CH₃CHBrCOOEt and activated zin dust and THF were added to the α-ketoester-harringtonine (at −78° C.) for 6 hours followed by slow warming to room temperature with stirred. The reaction mixture was diluted with 10 L CHCl₃ and 10 L H₂O and solid Na₂CO₃ was added. CHCl₃ was evaporated under reduced pressure and residue was obtained.

The residue was purified by chromatography on alumina. The column was flushed with chloroform and followed by chloroform-methanol (9:1). The solvents were recovered under reduced pressure to provide as a solid. Solid was dissolved in pure ethanol and crystallized. The crystals were refined by recrystalization in diethyl ether. The crystals dried under vacuum. The product is HHT, which has the following characters:

[α]_{D} −119° (C=0.96),

MSm/e (%): 689 (M⁺, 3), 314 (3), 299 (20), 298 (100), 282 (3), 266 (4), 20 (3), 150 (8), 131 (12), 73 (18)

### EXAMPLE 3

### HHT Extracted from Plant Tissue

Extraction of HHT has several major methods which including extraction by organic solvent, chromatograph and adjust pH.

HHT was extracted from plant tissue culture, plant cells or leaves of Cephalotaxus species.

1 kg of ground Cephalotaxus fortunei Hook was extracted with 10 liters of water at room temperature for 24 hrs. To filtered the solution to yield a filtrate. Ten liters of 90% ethanol added to filtrate. The mixture was Centrifugalized to yield a sediment. Percolated the sediment with ethanol and filter again to yield filtrate, combined filtrates, and distilled under reduced pressure to recover ethanol and an aqueous residue. To this residue, added 10% of HCl to adjust the pH to 2.5. To separated the solids from the solution by filtration to yield a filtrate (1). Washed the solids once with 2% HCl and filtered to yield a filtrate (2). Combined (1) and (2) and adjusted the pH to 9.5 by adding saturated sodium carbonate solution. Extracted the alkaline filtrate with chloroform and separated the chloroform layer from the aqueous layer. To repeated this extraction process five times. Combined all the chloroform extracts and distilled at reduced pressure to recover chloroform and alkaloid as a solid residue obtained. The solid alkaloid was then dissolved in 6% citric acid in water. The solution was divided into three equal portions. These were adjusted to pH 7, 8 and 9 by adding saturated sodium carbonate solution. The portions having pH 8 and 9 were combined and extracted with chloroform. The chloroform extracts were distilled under reduced pressure, whereby chloroform was removed and recovered and crude HHT was obtained. The crude HHT was dissolved in pure ethanol and crystallized. The crystals were refined by recrystallization in diethyl ether. The crude HHT obtained.

The portion having a pH of 7 passed through a liquid chromatographic column packed with alumina of diameter to height 1:50. The column was finally flushed with chloroform and followed by chloroform-methanol of 9:1 mixture. The resulting alkaloids were mixture crude of HHT. Combined crude HHT and then separated from each other by countercurrent distribution employing chloroform and pH 5 buffers. The first fraction of the countercurrent distribution was HHT. HHT was purified by crystallization in methyl alcohol. The crystallization was purified by recrystallization in methyl alcohol and dried under vacuum.

### EXAMPLE 4

### Effect of HHT on Cellular Diversion of Human Leukemic Cells

The present invention disclosed the new method for making HHT. The following examples described that pharmaceutical effects are same between HHT which manufacturing from traditional methods and HHT which manufacturing from new methods of the present invention. The term “C-HHT” and “S-HHT” when used here is to refer to HHT from culture and HHT from semisynthesis.

HL-60 cells were culture in a two-layer soft agar system for 10 days without adding any growth factors as described previously, and colonies were counted using an inverted microscope. The analogues were added to the agar upper layer on day 0. For analysis of the reversibility of inhibition of proliferation, the cells were cultured in suspension culture with and without HHT. After 60 hours, the culture flasks were gently jarred to loosen adherent cells, the cells were washed twice in cultured medium containing 10% FCS to remove the test drugs, and then the clonogenic assay was performed. NBT % indicated percentage of normal cells. N is Normal cells and C is Human leukemic cells. These results were periodically confirmed by fluorescence microscopy and by DNA fragmentation.

TABLE 1
Effect of drugs on cellular diversion of human leukemic cells

| Group | NBT (%) |
|---|---|
| N | 98 ± 12 |
| C | 5.0 ± 0.6 |
| HHT | 65 ± 7.0* |
| C-HHT | 64 ± 8.0* |
| S-HHT | 63 ± 7.8* |

*P < 0.01 compared with control group.
NBT (%) is index of normal cells. The higher NBT (%) means higher normal cells.

Data of Table 1 showed that HHT could significantly induce diversion of leukemic cells to normal cells. C-HHT and S-HHT are as similar as regular HHT.

### EXAMPLE 5

### Effect of HHT on Cellular Diversion of Gastric Cancer Cells

The gastric cancer cells and normal cells were cultured in PRMI 1640 medium supplement with 10% FCS serum. Other method is similar to example 4.

TABLE 2
Effect of HHT on diversion of gastric cancer cells

| Group | NBT (%) |
|---|---|
| N | 95 ± 14 |
| C | 8 ± 1.2 |
| HHT | 58 ± 8.9* |
| C-HHT | 57 ± 8.9* |
| S-HHT | 58 ± 8.1* |

*P < 0.01 compared with control group.

Data of Table 2 showed that HHT could significantly induce diversion of gastric cancer cells to normal cells. C-HHT and S-HHT are as similar as regular HHT.

### EXAMPLE 6

### Effect of HHT on Apoptosis of Cancer Cells

Methods

Human leukemia cells (HL-60) were grown in RPMI Medium 1640 supplemented with 10% (v/v) heat-inactivated FBS (56° C. for 30 min) at 37° C. in a humidified 95% air/5% CO₂ atmosphere. Cells were seeded at a level of 2×10⁵ cells/ml. Cells were allowed to attain a maximum density of 1.2×10⁶ cells/ml before being passed by dilution into fresh medium to a concentration of 2×10⁵ cells/ml.

Cell pellets containing 5×10⁶ cells were fixed with 2.5% glutaraldehyde in cacodylate buffer (pH 7.4), dehydrated through graded alcohol, and infiltrated with LX-112 epoxy resin. After overnight polymerization at 60° C. 1-μm sections were cut with glass knives using a microtome. The sections were stained with 1% toluidine blue and coverslipped. In addition, experimental examples were stained with May-Grunwald-giemsa stain for the demonstration of apoptosis.

Determination of Apoptosis

Method (1): Apoptosis Was Quantitated by Flow Cytometry

Cells (2.5×1060 were incubated in 10 ml IMDM plus 105 heat-inactivated fetal calf serum. Samples were incubated for 24 hours with various concentrations of drugs. Control samples received the same amount of media, without drug addition. After 24 hours of incubation the samples were pelleted and fixed in ethanol 705 for 15 minutes at 4° C.; after three washes in PBS, the cells were treated with RNase I 0.5 mg/ml for 15 minutes at 37° C. The cells were harvested by centrifugation and resuspended in 50 μg/ml propidium iodide in PBS. Analysis (upon acquisition of 10,000-20,000 events) was performed on a FACscan flow cytometer with the FL2 detector in logarithmic mode, using Lysis II software (Becton Dickinson). Apoptotic cells were located in the hypodiploid region of the histogram, due to chromosome condensation and fragmentation.

For evaluation of apoptosis by flow cytometry, cells were fixed and permeabilized in 1% paraformaldehyde and ice-cold 70% ethanol. Digoxigenin-dUTP was incorporated at the 3′OH ends of the fragmented DNA in the presence of terminal deoxynucleotidyltranserase, and the cells were incubated with FITC-labeled anti-digoxigenin-dUTP and with propidium iodide. Green (apoptotic cells) and orange (total DNA) fluorescence were measured with a FACScan flow cytometer and analyzed with LYSIS II and CELLFIT programs. Data were analyzed by Student's t-test. P values were considered significant when <0.05.

Method (2): DNA Electrophoresis

Untreated and treated HL-60 cells collected by centrifugation, washed in phosphate buffered saline and re-suspended at a concentration of 5×10⁶ cells and 0.1% RNase A. The mixture was incubated at 37° C. for 30 min and then incubated for an additional 30 min at 37° C. Buffer was added and 25 μl of the tube content transferred to the Horizontal 1.5% agarose gel electrophoresis was performed at 2 V/cm. DNA in gels visualized under UV light after staining with ethidium Bromide (5 μg/ml). DNA fragmentation assays: DNA cleavage was performed, quantitation of fractional solubilized DNA by diphenylamine assay and the percentage of cells harboring fragmented DNA determined by in labeling techniques. For the diphenylamine assay, 5×10⁶ cells were lysed in 0.5 mL lysis buffer (5 mmol/L Tris-HCl, 20 mmol/L DTA, and 0.5% Triton X-100, pH 8.0) at 4° C. Lysates were centrifuged (15,000 g) for separation of high molecular weight DNA (pellet) and DNA cleavage products (supernatant). DNA was precipitated with 0.5 N perchloric acid and quantitated using diphenylamine reagent. The cell cycle distribution was determined 4 hours after addition of drug and represents mean ±SD of 5 independent experiments.

Method (3)

Apoptosis of HL-60 cells were assessed by changes in cell morphology and by measurement of DNA nicks using the Apop Tag Kkt (Oncor, Gaithersburg, Md.). Morphologically, HL-60 cells undergoing apoptosis possess many prominent features, such as intensely staining, highly condensed, and/or fragmented nuclear chromatin, a general decrease in overall cell size, and cellular fragmentation into apoptotic bodies. These features make apoptotic cells relatively easy to distinguish from necrotic cells. These changes are detected on cytospin preparations stained with Diff-Quick Stain Set. Apoptotic cells were enumerated in a total of about 300 cells by light microscopy.

TABLE 3
Effect of HHT on apoptosis of cancer cells (1)

Apoptosis (%)

| | Normal cells | Human leukemia cells |
|---|---|---|
| No drug | 8.1 ± 2.0 | 4.0 ± 0.9 |
| HHT (10 ng/ml) | 16.5 ± 2.0* | 33.8 ± 4.8** |
| HHT (50 ng/ml) | 18.5 ± 2.5* | 42.8 ± 5.2** |
| HHT (100 ng/ml) | 22.0 ± 3.5* | 60.2 ± 8.9** |
| HHT (500 ng/ml) | 29.5 ± 5.2* | 92.0 ± 10.8** |

*P < 0.01 compared with group of normal cells.
**P < 0.01 compared with group of human leukemia cells

TABLE 4
Effect of HHT on apoptosis of malignant melanoma cells

Apoptosis (%)

| | Normal cells | Malignant melanoma cells |
|---|---|---|
| HHT (50 ng/ml) | 18.5 ± 2.5 | 42.8 ± 5.2 |
| C-HHT (50 ng/ml) | 19.2 ± 2.5* | 43.8 ± 4.2* |
| S-HHT (50 ng/ml) | 18.0 ± 2.0* | 43.0 ± 6.0** |

*P < 0.01 compared with group of human leukemic cells
**P < 0.05 compared with group of human leukemic cells

Data showed that HHT could significantly induce apoptosis of cancer cells. The significantly increasing apoptosis of HHT on human leukemia cells and eye tumor cells are showed in the Table 3. The effect of C-HHT and S-HHT is as same as regular HHT (Table 4).

### EXAMPLE 7

### Effects of HHT on Tumor Cells Proliferation

Materials and Methods

Human tumor cell lines: Hela leukemia HL-60, malignant melanocarcinoma B16, oral epidermoid carcinoma (KB), lung carcinoma (A549), breast carcinoma MCF-7, adenocarcinoma of stomach.

Animal tumor cell lines: Walker carcinoma, LLC-WRC-256, malignant melanoma (RMMI 1846), 3T3, and S-180 sarcoma (CCRF-180). All lines were routinely cultured in the RPMI1640 medium supplemented 20% fetal calf serum. The experiment was carried out in 96 microplate, each well had 5×10⁵ cells and given desired concentration of 1 μg/ml (1×10⁻⁶ g/ml) drug. Then the plate was incubated at 370° C. in an atmosphere of humidified air enriched with 5 percent carbon dioxide for 72 hours.

Inhibition percent rate of tumor cell proliferation was obtained according to the bellow formula.

$$Inhibition percent rate = \frac{Control - Test}{Control} \times 100 \%$$

Results

TABLE 5
Effect of HHT on inhibiting growth cancer cells

Inhibition (%)

| Group | Leukemic cells | Malignant melanoma of eyes* |
|---|---|---|
| No drug | — | — |
| HHT | 73.8 ± 8.1 | 80.0 ± 9.2 |
| C-HHT | 74.1 ± 10.8 | 83.1 ± 9.8 |
| S-HHT | 72.5 ± 11.6 | 82.5 ± 10.2 |

*Malignant melanoma of the pigmented layers of eyes

Data of Table 5 showed that HHT, C-HHT and S-HHT could significantly inhibit human cancer cells proliferation.

### EXAMPLE 8

### The Effect of HHT on the Growth of Transplanted Tumor

Experimental Procedure

Male mice, weight 20-22 g, were used in the experiment. 1×10⁷ tumor cells were injected to mouse and other drugs injected intraperitoneally began second day. All mice were sacrificed on the 12th day, isolated the tumor and weighed and calculated the inhibition rate of tumor weight.

Results

The effect of HHT on the growth of animal transplanted tumor as illustrated by the Table 6.20 mg/kg of other drugs could inhibit the growth of L615 transplanted tumor.

TABLE 6
Effect of HHT on inhibition of transplanted tumor

| Group | Inhibition (%) |
|---|---|
| No drug | — |
| HHT | 68 ± 7.2 |
| C-HHT | 67.8 ± 6.0 |
| S-HHT | 69.2 ± 7.0* |

*P < 0.01 compared with HHT group.

Data of Table 6 showed that HHT, C-HHT and S-HHT could significantly inhibit animal transplanted tumor. The effect of C-HHT and s-HHT is as same as regular HHT.

### EXAMPLE 9

### The Effect of HHT on Decreasing of Tyrosine Kinase

The development of cancer cells can be viewed as a defect in the normal process of differentiation and disorder balance between proliferation and maturation that occurs in normal cells. The expression of oncogenes plans a very important role in regulate cellular proliferation. The tyrosine kinase (TK) is a protein product of expression of oncogenes. The TK catalyze the transfer of phasphate from ATP to the hydroxyl residues on protein substrates. Activity of the TK is essential for the malignant transformation of cells.

In subsequent years, a number of oncogenes have been found to code for TK. Such as src, yes, fgr, abl, erbB, mos, neu,fms, fps, ros and sis are considered to act through tyrosine kinase activity. TK activity is strongly correlated with the ability of retroviruses to transform cells. Also, maturation with reduced TK activity has lower transforming efficiency. Transformation of the HL-60 leukemia cells causes the high TK activity. In fact, TK activity is enhanced in many human cancers, such as breast carcinomas, prostate cancer cells, colon cancers, and skin tumor. The results of a lot of experiments indicated that tyrosine phosphorylation is an important intracellular mediator of proliferation and differentiation. Mature of cells possess relatively low levels of TK activity. Similar TK activity is also related with the cellular receptors for several growth factors such as EGF, platelet-derived growth factor, insulin, and growth factor. In general, very low levels of TK are expressed in normal cells and high levels of TK are expressed in cancer cells. Many evidences have been accumulated that the dysfunction of cellular oncogenes is a cause of human cancers. Therefore, a drug, which inhibits the activity of TK, can provide a new way to overcome cancer. In other words, the development of effective inhibitors of TK can be used for the treatment of cancer.

Materials and Methods

[³²P]ATP and other isotopes were purchased form Amersham Corp. All other chemicals were reagent grade obtained from commercial suppliers.

Cells: L1210 and P388 cells were grown at 37° C. on medium RPMI-1640 without antibiotics and supplemented with 10% horse serum. Cultures were diluted daily to 1×10⁵ cells/ml with fresh growth medium. From a culture initiated with cells from ascitic fluid obtained from a mouse 5 days after implantation with in vivo-passage leukemia, a stock of ampoule containing 10⁷ cells/ml in growth medium plus 10% dimethyl sulfoxide was frozen and stored in liquid nitrogen. Cultures were started from the frozen stock and were passage for no more than 1 month.

L1210 and P388 cells were grown at 37° C. on medium RPMI-1640 supplemented with 10% calf serum, 10,000 unit/ml of Penicillin and 10,000 unit/ml of Streptomycin. 1×10⁶/ml cells were placed in culture with different concentrations of HHT. Then the cell suspension was incubated at 37° C. in a humidified atmosphere of 5% CO₂-95% air for the indicated time. Reactions were terminated by addition of 3 ml of cold Earle's buffer. Cells were lysed, precipitated with 10% trichloroacetic acid (TCA) and filtered onto glass fiber filters. The filters were washed with phosphate-buffered saline and placed in scintillation vials, and radioactive emissions were counted.

Tyrosine kinase (TK) Assay: TK was measured by a modification of the method of Braun et al. Briefly, H-60 leukemia cells were plated at a density of 5×10⁵ cells in 60-nm dished, and divided control and treatments groups for incubation 24 hours at 37° C. with 5% CO₂. The cells were collected by scraping, washed twice with phosphate-buffered saline, and resuspended at density of 10⁶ cells/ml in 5 mM HEPEs buffer (pH 7.4). The cells were then resuspended in 1 ml of buffer containing 5 mM HEPES (pH 7.6), 1 mM MgCl₂ and 1 mM EDTA, then placed on ice bath. The cell membrane was disrupted by ultra sound and centrifuged at 1,000×g for 10 minutes. The supernatant was ultra centrifuged at 30,000×g for 30 minutes at 4° C. The pellet was resuspended in 0.3 ml of buffer containing 25 mM HEOES, centrifuged at 12,000×g for 5 minutes. The resulting supernatant was used for TK assay. Content of protein was determined. 10 μg of protein placed in 20 mM HEPES (pH 7.6), 15 mM MgCl₂, 10 mM ZnCl₂ and 5% (v/v) nonidet P-40, with or without substrate [glutamic acid (GT), mg/ml]. After 5 minutes incubation at 25° C., the reaction was initiated by the addition of 25 μM [γ³²P] ATP (3 ci/mmol). After 10 minutes, the reaction was stopped by the addition of 20 mM cold ATP. 50 μl of the mixtures were spotted on glass microfiber filter discs and washed three times with cold trichloroacetic acid (TCA), contained 10 mM sodium pyrophosphate. Air dried. Radioactivity was determined by liquid scintillation spectrometry. The net TK activity was determined after correcting for endogenous TK activity.

Results and Discussion

The present study clearly demonstrated that HHT reduction in TK activity. A concentration-dependent inhibition was seen. HHT caused a relatively strong inhibition, with inhibition 99.9% occurring at a concentration of 10⁻⁶ M.

TABLE 7
Effect of HHT on TK activity of HL-60 leukemia cells

| Drugs | Concentration (M) | % of control activity |
|---|---|---|
| No drug | — | 100 |
| HHT (1) | 10⁻⁶ | 0.6 |
| HHT (2) | 10⁻⁷ | 21.5 ± 4.0 |
| HHT (3) | 10⁻⁸ | 87.8 ± 12 |

TABLE 8
Effects of HHT on TK activity

| Group | % of control activity |
|---|---|
| No drug | 100 |
| HHT | 21.5 ± 4.0 |
| C-HHT | 20.0 ± 5.0 |
| S-HHT | 20.8 ± 4.8** |

*P < 0.01 compared with HHT
**P < 0.05 compared with HHT

HHT has significantly inhibited TK activity (Table 7). HHT, C-HHT and S-HHT have same effect (Table 8).

### EXAMPLE 10

### HHT Inhibited Tumor Incidence in Vivo

The capacity of tobacco-specific nitrosamine 4-(methylnitrosamino)-1-(3-pyridyl)-1-butanone (NNK) to induce tumor incidence was recognized several years ago.

Methods:

Every group had 20 mice. For treatment group, each mouse was gave Drug by injection at dose of 20 mg/kg daily. For control group, each mouse was gave same volume of physiological saline. Three days later, mice were gave 10 μmol NNK (in 0.1 ml saline) by i.p. injection. Sixteen weeks after these treatments the mice were killed and pulmonary adenomas were counted. The statistical significance of bioassay data was determined by student's test.

TABLE 9
Effect of HHT on NNK-induced lung tumorigenesis

| Group | Tumor incidence (%) |
|---|---|
| No drug | 100 |
| HHT | 35.8 ± 4.5 |
| C-HHT | 21.7 ± 4.8* |
| S-HHT | 26.5 ± 4.9* |

*P < 0.05 compared with HHT group

Data of Table 9 indicated that HHT, C-HHT and S-HHT have a significant inhibitory effect against lung tumor and it means HHT could decrease tumor incidence. Therefore, HHT, C-HHT and S-HHT could prevent cancer. HHT, C-HHT and S-HHT also have same effect.

### EXAMPLE 11

### Toxicological Data (1)

1. LD₅₀ of HHT is 4.07 mg/kg injection in abdominal cavity in mice.
2. LD₅₀ of C-HHT is 4.0 mg/kg injection in abdominal cavity in mice.
3. LD₅₀ of S-HHT is 4.20 mg/kg injection in abdominal cavity in mice
4. LD₅₀ of HHT is much higher than majority anticancer drugs. The toxicology data of HHT mean that HHT is safe drug for treatment of cancer cells.

The LD₅₀ of HHT indicated that HHT is a safe anticancer drug.

### EXAMPLE 12

### Toxicological Data (2)

Forty dogs were used. Body weight, food consumption, general observations, laboratory tests, and postmortem examinations were determined.

Twenty normal adult dogs had weight approximately 10 kg±1 kg used in experiments. Toxic doses for dogs: 0.15 mg/kg/days×7 and 0.30 mg/kg/days×7 of HHT was injected as the toxic doses.

For HHT treatment group, at least eight dogs that died early in the observation period and eight dogs that died late were examined. After injection of lethal doses the major target organs involved in toxicity in dogs produced by 7 day treatments with HHT were limited to G.I. tract, heart and hematopoietic organs. Most deaths were caused by cardiac dysfunction. After injection of lethal doses, hepatic toxicities of mild to moderate degree occurred only in individual cases. When such treatments were repeated for two additional courses, no additional significant toxicity was observed. However, the cardiac and hematopoietic toxicity appeared to be moderately degree.

The data of short-term toxicity in dogs for HHT were summarized as the following table.

TABLE 10
Safety of HHT

| | HHT (dose) | | | | | |
|---|---|---|---|---|---|---|
| | 0.1 | | 0.2 | | 0.3 | |
| | mg/kg/d × 7 | | mg/kg/d × 7 | | mg/kg/d × 7 | |
| Reaction | 20 ♀ | 20 ♂ | 20 ♀ | 20 ♂ | 20 ♀ | 20 ♂ |
| Body weight (decreasing %) | 8.9 | 8.8 | 11.0 | 10.9 | 12.5 | 13.0 |
| Cardiovascular system | | | | | | |
| Heart rate (beat/min) | 208 | 205 | 232 | 230 | 260 | 259 |
| Abnormality of ECG, ST-T | − | − | ± | ± | + | + |
| Cardiac necrosis | − | − | − | − | ± | ± |
| Hematopoietic/hemostatic system | | | | | | |
| Hemoglobin % | 8.8 | 9.0 | 9.3 | 9.2 | 9.5 | 9.6 |
| Erythrocyte count (×10⁶/mm³) | 3.90 | 3.94 | 3.70 | 3.65 | 3.40 | 3.40 |
| Leukocyte count (×10³/mm³) | 3.91 | 3.90 | 3.65 | 3.58 | 3.25 | 3.20 |
| Platelet count (×10⁴/mm³) | 4.9 | 5.0 | 3.75 | 3.70 | 2.90 | 3.00 |
| Liver | | | | | | |
| Glutamic pyruvate transaminase (GPT) | 65 | 68 | 72 | 73 | 75 | 73 |
| Hepatic necrosis | − | − | ± | ± | + | + |
| Hepatic fibrosis and cirrhosis | − | − | − | − | ± | ± |
| Hepatic ecchymosis | − | − | − | − | ± | ± |
| Albumin-globulin ratio (A/G %) | 89 | 90 | 92 | 93 | 95 | 92 |
| Gastrointestinal system | | | | | | |
| Vomiting | + | + | + | + | ++ | ++ |
| Anorexia | + | + | ++ | ++ | +++ | +++ |
| Diarrhea | ± | ± | + | + | + | + |
| Marrow | | | | | | |
| Marrow depression | + | + | + | + | ++ | ++ |
| Renal/urinary system | | | | | | |
| Renal necrosis | − | − | − | − | ± | ± |
| Renal toxic nephropathy | − | − | − | − | ± | ± |
| Spleen | | | | | | |
| Spleen necrosis | − | − | − | − | ± | ± |
| Reproductive system | | | | | | |
| Seminal depression | − | − | − | − | − | − |

“±”: either positive or negative;
“+”: positive, light;
“++”: middle,
“+++”: heavy
Normal data of index are listed below.
Hemoglobin: 11.0~13.5 g;
erythrocyte count: 4.11~5.03 × 10⁶/mm³;
leukocyte count: 5.6~10.9 10³/mm³;
Platelet count: 11.2~34.8 × 10⁴/mm³;
NPN: <46.0 mg %;
GPT: <49%;
A/G: 47~65/35~52;
heart rate: 240 beat/min.

All toxicities observed were dose-dependent, completely reversible upon discontinuation of treatment. No significant delayed toxicity was noted during an observation period of 6 weeks or more. No sex related differences in qualitative toxicity of HHT were observed.

In conclusion, toxicity of HHT is low.

### EXAMPLE 13

### Analysis of Chromosomes

For metaphase chromosomes, kidney cell cultures were treated with colchicines (0.4 μg/ml) for 3-4 hours. The cells were then trypsinized and treated with hypotonic solution (0.075 M KCl) at 37° C. for 30 minutes. The cell suspensions were centrifuged and the pellets fixed in cold acetic acid:methanol (1:3) solution. Slides were prepared by standard air-drying method and stained with 2% Giemsa solution. The results scored by analyzing at least 200 well spread metaphases with 44±2 chromosomes for gaps, chromatid and chromosome breaks and exchanges, and association. Chromatid and chromosome aberrations were scored separately, and the total percentage was subjected to statistical analysis. Gaps were recorded but not included in the total frequency. Endoreduplication (endomitosis) was estimated from at least 500 cells/animal and expressed as a percentage.

TABLE 11
Chromosomal aberrations induced by HHT in kidney

| | | |
|---|---|---|
| | Aberration/100 cells | |
| Duration of treatment | Chromosome | % Aberrant cells |

| | | | |
|---|---|---|---|
| (months) | Breaks | Exchanges | (mean ± SEM) |

Untreated

| | | | |
|---|---|---|---|
| 0.5 | nd | nd | 0.2 ± 0.1 |
| 1.0 | nd | nd | 0.3 ± 0.2 |
| 2.0 | nd | nd | 0.3 ± 0.1 |
| 3.0 | 0.1 | nd | .0.5 ± 0.3 |
| 4.0 | nd | 0.1 | 0.5 ± 0.2 |
| 5.0 | 0.1 | nd | 0.4 ± 0.2 |

HHT

| | | | |
|---|---|---|---|
| 0.5 | 0.1 | nd | 0.5 ± 0.1 |
| 1.0 | 0.5 | nd | 0.8 ± 0.2 |
| 2.0 | 0.7 | 0.1 | 1.2 ± 0.4 |
| 3.0 | 0.9 | nd | 1.8 ± 0.7 |
| 4.0 | 1.2 | 0.1 | 2.6 ± 0.8 |
| 5.0 | 1.3 | 0.2 | 5.5 ± 0.5 |

The data of Table 11 indicated that HHT has no exchange in chromosome, no chromatid or chromosome aberrations and no significant differences in the frequency of either chromosome lesions or chromatid or chromosome aberrations with increasing age.

### EXAMPLE 14

### Mutagenic Effect of HHT

Determination of the mutagenic and carcinogenic activity is important for estimating side effects of drug. The mutagenic activity of many drugs can only be detected with growing cells. In present study, mutagenic and carcinogenic activity of HHT is determined by Bacteria system.

The method for detecting mutagenicity of HHT, with the Salmonella system that detects the reversion of the bacteria from His⁻ to His⁺, is widely used.

Methods for detecting carcinogens and mutagens with the salmonellia mutagenicity test are highly efficient in detecting carcinogens and mutagens. Major carcinogens tested have been detected as mutagens. Salmonella mutagenicity assay is very sensitive and simply test for detecting mutagens and carcinogens. Therefore, it has been useful in a detailed study that has been made of mutagenic activity of HHT.

TAa7, TAa8, TA100 and TA102 are extremely effective in detecting classes of carcinogens and mutagenesis.

Methods

The bacterial tester strains used for mutagenesis testing are TA97, TA98, TA100 and TA102. Mutagenesis testing method was done as described previously ^{[77-88]}. In brief, TA97, TA98, TA100 and TA102 were grown in agar gel culture. The petri plats (100×15 mm style) contain 30 ml with 2% glucose. The agar mixture was agitated vigorously and immediately poured into plates of minimal agar. The cultures were incubated at 37° C. in a dark and 5% CO₂ in air for 48 hours. After 48 hours the colonies in both test and controls are counted. The presence of a background lawn of bacteria on the histidine-poor soft agar plate was used as an indication that gross toxic effects were absent. Mutagenicity assays were carried out at least in triplicate.

Results and Discussion

The data of experiment summarized as the following table.

TABLE 12
Mutagenesis Assay on plates

| | Dose/ | Number of His⁺ revertants/plate | | | | | |
|---|---|---|---|---|---|---|---|
| | plate | TA97 | | TA98 | | TA100 | |
| Treatment | (μg) | −S | +S | −S | +S | −S | +S |
| Spontaneous | — | 149 ± 15 | 150 ± 17 | 35 ± 4 | 36 ± 4 | 120 ± 17 | 120 ± 15 |
| 4NQO | 0.5 | 861 ± 79 | — | 338 ± 35 | — | 2301 ± 190 | — |
| HHT | 100 | 150 ± 16 | 160 ± 17 | 32 ± 4 | 34 ± 4 | 158 ± 15 | 160± |
| HHT | 10 | 160 ± 16 | 165 ± 16 | 38 ± 4 | 36 ± 3 | 162 ± 17 | 165± |
| HHT | 1 | 130 ± 11 | 150 ± 14 | 30 ± 3 | 32 ± 4 | 140 ± 13 | 152± |
| HHT | 0.1 | 120 ± 10 | 145 ± 14 | 29 ± 3 | 34 ± 4 | 148 ± 12 | 158± |

*4QO: 4-nitroquinoline-1-Oxide
The salmonella typhimurium strains TA97, TA98 and TA100 were checked using 4-nitroquinoline-1-oxide. The range of spontaneous mutation rates for the individual strains, which were considered to be acceptable, was TA97 (100-170), TA98 (20-40) and TA100 (80-150).

The data of Table 12 indicated that the number of His+ revertants/plate of HHT almost is as same as spontaneous of testing strains. On the contrary, 4NQO is mutagenic and carcinogenic agent. The number of His+ revertants/plate of 4NQO is higher than 10 times of spontaneous.

In conclusion, HHT has no carcinogenic and mutagenic action.

### EXAMPLE 15

### Metabolism of [³H]-HHT

The data of metabolism of HHT provide a vital tool for understanding of HHT action including how many HHT gets to the sites where it activates pharmacological and chemotherapeutic activity and how long it remains there. The data above also help us to understand the amounts of HHT in organs and metabolites in the major organs. Metabolism of HHT is established to quantitatively dynamic processes. In present study, methods of [³H]-HHT used for determined HHT. So far, it is best methods for research metabolism of HHT.

Methods

Animals: Adult DBA/2J male mice, 6 to 8 weeks old. All animals weighed approximately 25 g when used in experiments. Each member of which received identical dosed (i.p.) of HHT.

Tumor: P388 leukemic cells induced in mice by inoculation with L1210 or P388 leukemic cells (1.0×10⁵).

After P388 induced in mice 5 days, 1 mg/kg of [³H]-HHT (100 μC/mg) was injected to mice. Volumes were 0.01 ml/g body weight. Mice were killed at 1, 3, 6 and 24 hours after injection of [³H]-HHT. The key data of HHT in metabolism process are determined counts per minute (CPM) of [³H]-HHT in organs. CPM is determined by liquid scintillation counting (LSC). The methods of LSC combined with radioactive of [³H]-HHT can be determined using a tracer of HHT in organs. After killed animals, organs and blood were obtained from P388 mice. Organs were weighted. 10 mg of organs were digested. 10 ml of scintillation solution (which containing 0.5% and 0.00% of POPOP) were added to digested solution and determined at LSC. Other processes are similar to the standard method.

Results and Discussion

CPM of [³H]-HHT of different organs is listed as the following table:

TABLE 13
Metabolism of HHT

| | CMP/mg organ | | | |
|---|---|---|---|---|
| Organs | 1 hour | 3 hours | 6 hours | 24 hours |
| Brain | 499 | 646 | 793 | 986 |
| Heart | 1403 | 1800 | 1193 | 893 |
| Liver | 4145 | 6718 | 4109 | 2175 |
| Spleen | 2828 | 2426 | 2198 | 2156 |
| Lung | 2100 | 2427 | 1788 | 1312 |
| Kidney | 6501 | 3121 | 3501 | 1065 |
| Intestine | 1638 | 3288 | 2475 | 1067 |
| Stomach | 1626 | 2321 | 3703 | 1823 |
| Bone | 500 | 560 | 505 | 480 |
| Contents of intestine | 3323 | 7215 | 6250 | 5009 |
| Contents of stomach | 6821 | 84375 | 100719 | 117914 |

TABLE 14
[³H]-HHT in blood

| | 1 h | 2 h | 4 h | 8 h | 16 h | 24 h |
|---|---|---|---|---|---|---|
| CPM | 1820 | 1650 | 1100 | 920 | 428 | 150 |

The data of Table 13 showed that after injected 1 hour, [³H]-HHT came to kidney, liver and spleen; after injected 3 hours, liver had highest CPM. The data of Table 14 indicated that CPM of [³H]-HHT was markedly decreasing in blood system. CPM of major organs is significantly decreasing after 2 hours. However, CPM of bone is slowly changed.

In conclusion, the data of metabolism of HHT showed the HHT could safely be used as a drug.

### EXAMPLE 16

### Preparation of HHT

HHT can be used with a traditional pharmaceutical excipient, diluent or carrier. HHT also can be used with cream or an aqueous solution for treatment of ophthalmologic disease.

Dose: HHT was administered at a dose of 1˜5 mg/m² as infusion through venous or injection.

What is claimed as new and desired to be protected by Letter Patent is set forth in the appended claim:

1. A method of treating cancer disease containing Homoharringtonine (HHT) which prepared by the process of semi-synthesis comprising:
(a) extracting Cephalotaxus (CEP) from culture cells and plant tissue or natural plant material of Cephalotaxus species; and
(b) semi-synthesis of HHT from CEP.

2. A process for producing HHT in accordance with claim 1 wherein said extracting CEP comprising:
(a) extracting a ground cultured plant tissue or plant selected from the group consisting of Cephalotaxus fortunei Hook, C. sinensis Li, C. hainanensis, C. wilsoniana and other Cephalotaxus species with 90% ethanol at room temperature for 24 hours;
(b) the ethanol was concentrated under reduced pressure;
(c) tartaric acid was added to concentrated ethanol solution;
(d) ammonia water was added to acidic solution and adjusted pH to 9;
(e) pH 9 solution was filtered and yielded filtrate;
(f) filtrate was extracted with CHCl₃;
(g) CHCl₃ was recovered and residue was obtained;
(h) residue was chromatographed packed with alumna and eluted by CHCl₃-MeOH;
(i) elute was concentrated under reduced pressure and residue was dried under vacuum; and
(j) the dried residue is Cephalotaxus (CEP), which used for semi-synthesis of HHT.

3. The method of claim 1 and 2, wherein said semi-synthesis of HHT from CEP comprising:
(a) benzene-α-acetone-Na was put into benzene;
(b) mixture was stirred then was dissolved in pyridine at stirred at 0° C.;
(c) oxalic chloride was added to solution of pyridine;
(d) solution warmed to room temperature and stand overnight;
(e) the solution was added to CH₂Cl₂ and cooled to 0° C.;
(f) CEP and pyridine were added to cold CH₂Cl₂ solution;
(g) mixture (1) was washed with 10% Na₂CO3 and saturated NaCl solution;
(h) mixture (1) was evaporated and solid α-ketoester-harringtonine obtained;
(i) CH₃CHBrCooEt and activated zin dust were added to α-ketoester-harringtonine and mixture (2) was obtained;
(j) CHCH₃ and H₂O and solid Na₂CO₃ were added to the mixture (2);
(k) Mixture (2) distilling under reduced pressure to recover CHCl₃ and residue was obtained;
(l) the residue was chromatography picked with alumina;
(m) column eluted with chloroform and followed by chloroform-methanol;
(n) solvents were recovered under reduced pressure and solid was obtained;
(o) solid was dissolved in ethanol;
(p) ethanol was recovered under reduced pressure and crystals were obtained;
(q) crystals were recrystallized in diethyl ether;
(r) crystals were dried under vacuum; and
(s) the product is HHT.

4. A safe anticancer drug HHT, according to claim 1, wherein said HHT from seni-synthesis has same pharmaceutical and toxicologic characters with HHT extracted from natural plant.

5. The method of isolating homoharringtonine (HHT) and harringtonine (HT) from leaves of Cephalotaxus species comprising an anti-gastric cancer cells agent, an induce apoptosis of cancer cells agent, an inhibiting tumor cells proliferation agent, inhibiting growth of transplanted tumor agent, decreasing of tyrosine kinase of cancer cells agent, and inhibiting tumor incidence agent.

6. The method of claim 5, isolating HHT and HT from leaves of Cephalotaxus species, further comprising the steps of
(a) extracting a ground cultured cells or plant tissue selected from the group consisting of Cephalotaxus fortunei Hook, C. sinensis Li, C. hainanensis and C. wilsoniana or other Cephalotaxus species with water at room temperature for 24 hours;
(b) water solution was filtered and filtrate obtained;
(c) 90% of ethanol added to filtrate;
(d) the mixture was centrifuigalized and sediment obtained;
(e) percolating the sediment with ethanol and collecting a filtrate;
(f) filtrates distilling under reduced pressure to recover ethanol and a residue obtained;
(g) adjusting the pH of the residue to 2.5;
(h) separating solids from the resulting mixture by filtration to yield a filtrate;
(i) adjusting the pH of the filtrate of to 9.5;
(j) extracting the alkaline solution five times with chloroform, combining all the chloroform extracts and distilling them to recover chloroform and alkaloids obtained;
(k) dissolving the alkaloids in citric acid, dividing the solution into three portions, and adjusting the pH of the three portions to 7, 8, and 9;
(l) extracting the portions of pH 8 and 9 with chloroform;
(m) distilling the chloroform extract to yield raw homoharringtonine and harringtonine;
(n) purifying said harringtonine by crystallizing the same in pure ethanol and recrystallizing the same in diethyl ether;
(o) combining the portion of pH 7 and the mother liquors resulting from step (n);
(p) passing the solution of step (o) through a chromatographic column packed with alumina, flushing said column with chloroform and subsequently with a chloroform-methanol mixture to yield a mixture of harringtonine and homoharringtonine;
(q) separating the homoharringtonine from harringtonine by countercurrent distribution with chloroform and pH 5 buffer. The methyl alcohol added to first fraction;
(r) the mixture was concentrated under reduced pressure and crystallization is obtained;
(s) the crystallization was purified by recrystallization in methyl alcohol; and
(t) the crystal was dried in vacuum; and
(u) the final product was HHT.

7. A safe anticancer drug HHT and HT, according to claim 5, wherein said HHT and HHT extracted from leaves of Cephalotaxus species, had same pharmaceutical and toxicologic characters with HHT and HT extracted from bark of Cephalotaxus species.

8. A method for treatment of cancer disease, comprising HHT and HT in claims 1, 2, and 6, wherein said HHT and HT were safe agents with high LD50.

9. A method for treatment of cancer disease, comprising HHT and HT in claim 1, 2, and 6 for the treatment of the following conditions:
(a) Leukemia;
(b) Gastric cancer, and
(c) Ophthalmologic disease.

10. A safe pharmaceutical composition, according to claim 1, 2, and 6, in from of HHT in saline (1-5 mg/M²) for intravenous injections.

11. A safe pharmaceutical composition, according to claim 1, 2, and 6, which is prepared in unit dosage form.

12. A safe pharmaceutical composition, according to claim 1, 2, and 6, which in form of a saline solution or cream for treatment of ophthalmologic disease.

13. A safe anticancer natural drug, according to claim 1, 2, and 6, wherein said [³H]-HHT using for determination metabolism of HHT.

14. A safe anticancer natural drug, according to claim 1, 2, and 6, wherein said the data of metabolism of HHT show that the HHT could safely be used as a drug.

15. A method of treating cancer disease comprising Homoharringtonine (HHT) prepared by extracting the culture plant tissue and cells of Cephalotaxus sinensis Li or Cephalotaxus hainanensis Li or Cephalotaxus fortune Hook, or other Cephalotaxus species comprising the steps of:
(a) parts of stems, leaves, skins or roots of Cephalotaxus species are surface disinfected by treated in 70% ethanol for 10 minutes and followed by 0.1 HgCl₂ for 3 minutes;
(b) plant materials are washed five times for 10 minutes each by sterilized water;
(c) parts of plant are cut into small pieces (0.5˜1 mm) and put pieces to medium and supplemented with a new active ingredient of phylum mycota (IPM) precursor of HHT, naphthalene-acid (NAA), phenylolanine, tyrosine, kinetin and sucrose (MS medium);
(d) pH of medium is adjusted to 5.7˜5.8;
(e) agar is added to medium;
(f) callus tissues are collected from agar media and suspension cultured cells are harvested by filtration and cultured in MS medium;
(g) cultures are kept in culture room at 26° C.;
(h) friable callus tissues are obtained;
(i) callus tissues are inoculated into MS medium containing IPM, precursor of HHT, NAA, kinetin and surcrose;
(j) callus tissues are subcultured at 26° C. for 35 days on rotary shaker operated at 80 rpm;
(k) cells are subcultured into fresh medium of same composition every 2 weeks and maintained at 120 rpm at 26° C.;
(l) packed cell volume (PCV), fresh weight (FW), dry weight (DW), concentration of HHT and concentration of sugar are determined every 5^{th} day;
(m) cells are harvested and dried.

16. A safe anticancer drug HHT, according to claim 15, wherein said a method of preparation of Homoharringtonine (HHT) from culture cells and plant tissue comprising the steps of:
(a) extracting a ground cultured cells or plant tissue selected from the group consisting of Cephalotaxus fortunei Hook, C. sinensis Li, C. hainanensis and C. wilsoniana or other Cephalotaxus species with water at room temperature for 24 hours;
(b) water solution was filtered and filtrate obtained;
(c) 90% of ethanol added to filtrate;
(d) the mixture was centrifugalized and sediment obtained;
(e) percolating the sediment with ethanol and collecting a filtrate;
(f) filtrates distilling under reduced pressure to recover ethanol and a residue obtained;
(g) adjusting the pH of the residue to 2.5;
(h) separating solids from the resulting mixture by filtration to yield a filtrate;
(i) adjusting the pH of the filtrate of to 9.5;
(j) extracting the alkaline solution five times with chloroform, combining all the chloroform extracts and distilling them to recover chloroform and alkaloids obtained;
(k) dissolving the alkaloids in citric acid, dividing the solution into three portions, and adjusting the pH of the three portions to 7, 8, and 9;
(l) extracting the portions of pH 8 and 9 with chloroform;
(m) distilling the chloroform extract to yield raw homoharringtonine and harringtonine;
(n) purifying said harringtonine by crystallizing the same in pure ethanol and recrystallizing the same in diethyl ether;
(o) combining the portion of pH 7 and the mother liquors resulting from step (n);
(p) passing the solution of step (o) through a chromatographic column packed with alumina, flushing said column with chloroform and subsequently with a chloroform-methanol mixture to yield a mixture of harringtonine and homoharringtonine;
(q) separating the homoharringtonine from harringtonine by countercurrent distribution with chloroform and pH 5 buffer. The methyl alcohol added to first fraction;
(r) the mixture was concentrated under reduced pressure and crystallization is obtained;
(s) the crystallization was purified by recrystallization in methyl alcohol; and
(t) the crystal was dried in vacuum; and
(u) the final product was HHT.

17. A safe anticancer natural drug, according to claim 1, 2, and 15, wherein said HHT has no carcinogenic and mutagenic action.

18. A safe anticancer drug HHT, according to claim 1, 2, and 15, wherein said HHT extracted from culture cell has same pharmaceutical and toxicological characters with HHT extracted from natural plant.
